(19) United States
(12) Patent Application Publication
(10) Pub. No.: US 2016/0185879 A1
(43) Pub. Date: Jun. 30, 2016

(54) MODIFIED STARCH

(71) Applicant: BIOPOLYMER NETWORK LIMITED, Rotorua (NZ)

(72) Inventors: Jafar AL-HAKKAK, Auckland (NZ); Fadia AL-HAKKAK, Auckland (NZ); Nigel LARSEN, Christchurch (NZ)

(21) Appl. No.: 14/910,999

(22) PCT Filed: Aug. 8, 2014
(86) PCT No.: PCT/IB2014/063788

§ 371 Date: Feb. 8, 2016

(30) Foreign Application Priority Data
Aug. 8, 2013 (AU) 2013902978

(51) Int. Cl. C08B 31/00; C09K 8/08; A61Q 19/00; A61K 8/04; C08B 31/12; A61K 8/73

(52) U.S. Cl. CPC C08B 31/006; C08B 31/12; A61K 8/732; A61Q 19/00; A61K 8/042; C09K 8/08

(57) ABSTRACT

The invention relates to a modified starch product produced by cross-linking and carboxyalkylating starch. The modified starch is soluble in cold water and forms a clear viscous gel when hydrated. The gel has many applications including as an additive for use the drilling industry.

### 1. FIELD OF THE INVENTION

The invention relates generally to a modified starch product prepared by cross-linking and carboxyalkylating starch. The modified starch is able to form a gel on hydration with cold water.

### 2. BACKGROUND

Drilling fluid is an essential component of many aspects of geotechnical engineering, such as drilling for oil and gas. Liquid drilling fluids or drilling muds are added to the wellbore to facilitate the drilling process by suspending cuttings, stabilising exposed rock and providing buoyancy. Drilling muds provide hydrostatic pressure to prevent the fluids formed during the drilling process from entering the wellbore. They also cool the drill bit during drilling and carry out the drill cuttings.

Generally, a drilling mud should be of sufficient viscosity to prevent cuttings from settling to the bottom of the wellbore. Fluids that have shear-thinning properties are preferred. This allows the fluid to have a liquid consistency when drilling is occurring but a more solid consistency when drilling has stopped.

Water-based drilling muds generally consist of clays such as bentonite clay and additives like barium sulphate, calcium carbonate or hematite.

Thickeners are also used to influence the viscosity of the drilling mud, for example, xanthan or guar gums, carboxymethylcellulose or modified starches.

The best drilling mud will vary with the particular drilling application. As deeper and more challenging wells are being drilled, more is being demanded of drilling muds. Also, many of the ingredients used in drilling muds, such as natural gums, are rapidly increasing in price. Therefore, there is a need for alternative drilling muds and mud components. It is an object of the invention to at least go some way towards fulfilling this need.

### 3. SUMMARY OF THE INVENTION

Starch lubricant compositions comprising jet-cooked mixtures of starch, water and lubricants such as olefins, esters and polybutenes have been used as components in drilling muds.

The inventors have now surprisingly discovered that certain modifications made to starch will produce a product with ideal properties for use in drilling muds and many other applications.

In a first aspect the invention provides a cross-linked carboxyalkyl starch that forms a gel on hydration with cold water.

In one embodiment, the cross-linked, carboxyalkylated starch has a relative viscosity that is more than 30 times greater than the relative viscosity of the native starch; preferably more than 40 times greater; more preferably, more than 100 times greater.

In one embodiment, the cross-linked, carboxyalkylated starch has a storage modulus G′ that is more than 50 times greater than the G′ of the native starch; preferably more than 75 times greater; more preferably more than 100 times greater. In one embodiment the cross-linked carboxymethylated starch has a loss modulus G″ more than 30 times greater than the G′ of the native starch; preferably more than 40 times greater; more preferably more than 50 times greater.

In one embodiment, the cross-linked carboxyalkyl starch is carboxymethyl starch.

In a second aspect the invention provides a process for making a cross-linked carboxyalkyl starch that forms a gel on hydration with cold water, the process comprising the steps of:
(a) adding a cross-linking reagent to a suspension of starch in water at about pH 11.0 to 11.8;
(b) stirring the suspension at about room temperature until cross-linking has occurred, wherein the suspension is substantially free of anti-gelatinization salts;
(c) removing the solvent to produce a wet cross-linked starch product comprising about 25 to about 40 wt % water;
(d) mixing the wet cross-linked starch product with a C₁-C₃ alcohol at pH above about 11 such that the ratio of C₁-C₃ alcohol to water is about 90:10 to about 70:30 v/v;
(e) heating the mixture to about 50° C. to about 70° C. with one or more carboxyalkylating agents in a ratio of about 15 to about 35% wt % relative to the starch, until gelatinization occurs;
(f) cooling to room temperature and neutralising the solution to about pH 6 to 7;
(g) recovering the cross-linked carboxyalkyl starch product from the reaction mixture; and
(h) optionally, drying the product to provide a powder.

In one embodiment, the modified starch of the invention is derived from one or more starches selected from the group comprising potato, maize, rice, tapioca, wheat, arrowroot, sweet potato, barley, sago, amaranth, canna, pea, oat, rye, triticale and sorghum starch, as well as low amylose (waxy) and high amylose varieties thereof. Preferably, the modified starch of the invention is derived from potato or tapioca starch.

In one embodiment, about 0.5 wt % to 2 wt % of the cross-linking reagent is added, relative to the starch. Preferably, 1.0 to 1.5 wt % cross-linking reagent is added.

In one embodiment, the cross-linking reagent is selected from the group comprising sodium trimetaphosphate (STMP), sodium tripolyphosphate (STPP), epichlorohydrin (EPI) and mixtures thereof. Preferably, the cross-linking reagent is STMP or a mixture of STMP and STPP.

In one embodiment, the cross-linking reaction of step (b) is carried out for about 1 to 7 hours, preferably about 3 to about 6 hours, more preferably about 5 hours.

In one embodiment the carboxalkylating agent is chloroacetic acid.

In one embodiment, the wet cross-linked starch product is mixed with a C₁-C₃ alcohol at above pH 12, preferably above pH 13.

In one embodiment, the mixture containing the carboxyalkylating agent is heated at about 55 to about 70° C., preferably 65° C.

In one embodiment, the ratio of C₁-C₃ alcohol to water is about 85:15 to about 75:25, more preferably 80:20.

In one embodiment the C₁-C alcohol is selected from the group comprising ethanol, methanol and isopropanol, preferably ethanol or isopropanol.

In a third aspect, the invention provides a cross-linked carboxyalkyl starch produced by the process of the invention.

In one embodiment, the cross-linked carboxyalkyl starch of the invention is able to form a gel when mixed in water at a concentration of about 0.5 to about 2.0 wt %, preferably about 1 to about 1.5 wt %.

In a fourth aspect, the invention provides a gel comprising a cross-linked carboxyalkyl starch of the invention.

In one embodiment the gel comprises about 0.1 to 2.8 wt % cross-linked carboxyalkyl starch, preferably about 0.5 to 1.5 wt %, more preferably about 1 wt %.

In one embodiment, the gel comprises 0.1 to 2.8 wt % cross-linked carboxymethyl starch.

In one embodiment, the gel is clear.

In a fifth aspect, the invention provides the use of a gel of the invention in the manufacture of a cosmetic lotion. In one embodiment, the cosmetic lotion comprises about 0.5% gel of the invention.

### 4. BRIEF DESCRIPTION OF THE FIGURES

Embodiments of the invention will now be described with reference to the drawings in which:

FIG. 1 is a graph showing the relative viscosity of various gums including a gel of the invention (BPN-gum).

FIG. 2 is a graph showing the storage modulus of various gums including a gel of the invention (BPN-gum).

FIG. 3 is a graph showing the loss modulus of various gums including a gel of the invention (BPN-gum).

FIG. 4 is a graph showing the flow curves (shear stress vs shear rate) of various gums including a gel of the invention (BPN-gum) at 20° C.

FIG. 5 is a graph showing the flow curves (viscosity vs shear rate) of various gums including a gel of the invention (BPN-gum) at 20° C.

FIG. 6 is a graph showing the flow curves (shear stress vs shear rate) of various gums including a gel of the invention (BPN-gum) at 75° C.

FIG. 7 is a graph showing the flow curves (viscosity vs shear rate) of various gums including a gel of the invention (BPN-gum) at 75° C.

### 5. DETAILED DESCRIPTION OF THE INVENTION

### 5.1 Definitions

As used herein, the term “cold water” means water that is at room temperature, i.e., not hot water.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprised” are to be interpreted in similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### 5.2 the Cross-Linking Process

In one aspect the invention provides a process for making a modified starch that forms a gel on hydration with cold water. The starch is first cross-linked before being carboxyalkylated, preferably carboxymethylated.

Cross-linking of starch is a well-known means of changing its properties. A common type of cross-linking is via phosphoryl groups to form distarch phosphate diesters. Cross-linking agents such as STMP, STPP and EPI are also routinely used. However, the reaction generally takes place at about 30 to 50° C. in the presence of salts such as sodium sulphate and/or sodium chloride. These salts (anti-gelatinization salts) are thought to prevent gelatinisation of the starch which can occur at high pH (above 12) and at temperatures above 30° C.

To carry out the cross-linking reaction in the method of the invention, a suspension of starch is first prepared, for example, by mixing 100 g of starch with 150-400 g of water. The pH of the suspension of starch may be raised using a base such as NaOH and lowered to stop the reaction using HCl. In the process of the invention, the pH should be between about 11.0 and about 11.8, more preferably about 11.6.

One or more cross-linking reagents is added to the starch suspension, either before or after the base is added. In one embodiment, about 0.5 wt % to 2 wt % of the cross-linking reagent is added, relative to the starch. Preferably, 1.0 to 1.5 wt % cross-linking reagent is added. The cross-linking reagent can be any suitable reagent known in the art. If the modified starch is to be used in the cosmetic or food application, the cross-linking agent should be one that is suitable for use in such a product.

In one embodiment, the cross-linking reagent is selected from the group comprising STMP, STPP, EPI and mixtures thereof.

The reaction mixture is stirred for a time sufficient time to provide the desired degree of cross-linking. Generally this is from about 1 to 7 hours, preferably about 3 to 6 hours, more preferably, about 5 hours. The resulting cross-linked starch will generally contain at least 0.2% bound phosphorus by weight of starch, although ranges of 0.1-0.4% are acceptable.

As will be appreciated by a person skilled in the art, the degree of cross-linking can be increased by increasing the reaction time and/or the amount of cross-linking reagent used.

Where the reaction time is shorter, less cross-linking will occur, giving a product that produces a less viscous gel.

However, the reaction mixture should not be heated during cross-linking. Without being bound by theory, it is believed that the unique properties of the modified starch of the invention at least partly depend on the starch not gelatinising during cross-linking. Comparative studies show that when cross-linking occurs at higher temperatures, product is produced that cannot form a gel in cold water.

Anti-gelatinization salts raise the gelatinization temperature of the starch allowing a suspension of starch to be heated to higher temperatures without causing the starch granules to swell and rupture, leading to a gelatinized product. Examples of anti-gelatinization salts include but are not limited to sodium sulphate, sodium chloride.

However, the process of the invention omits these agents. Again, comparative studies have shown that the presence of these salts in the reaction mixture leads to a cross-linked carboxyalkyl starch that does not form a gel on hydration with cold water. This result is found even if the process of the invention is modified by isolating and washing the cross-linked starch prior to carboxyalkylation.

Therefore, a key step in the process of the invention is cross-linking the starch under conditions that prevent gelatinization, but without anti-gelatinization salts.

The wet cross-linked starch is then recovered from the reaction mixture by any technique known in the art, for example, filtration.

### 5.3 the Carboxyalkylating Process

The second part of the process of the invention is carboxyalkylation of the wet cross-linked starch.

The wet cross-linked starch is first suspended in C₁-C₃ alcohol at high pH. Generally, the pH should be above 11 but higher pHs are preferred, such as pH 12, 13 and 14. The pH can be raised using any suitable base, for example, NaOH.

The C₁-C₃ alcohol is critical to the process of the invention. While methanol and isopropanol can be used, ethanol is preferred. The C₁-C₃ alcohol must be added to the wet cross-linked starch so as to achieve a ratio of alcohol to water of about 90:10 to about 70:30 w/w. Preferably, the ratio of alcohol to water is about 85:15 to about 75:25 w/w, more preferably about 80:20.

To calculate the correct alcohol to water ratio, the amount of volume remaining in the wet cross-linked starch must first be determined. This can be achieved by calculating the difference between the volume of the initial reaction mixture and the volume of solvent removed during recovery of the wet cross-linked starch product.

Water present in the C₁-C₃ alcohol should be accounted for when calculating the ratio. For example, when 95% ethanol is used, a mixture of 82 ml ethanol to 18 ml water gives an overall ratio of ethanol to water of 80:20.

A carboxyalkylating agent is then added to the mixture of wet cross-linked starch in C₁-C₃ alcohol. Suitable carboxyalkylating agents are known in the art. In one embodiment, a carboxymethylating agent is used, for example, chloroacetic acid, dichloroacetic acid, trichloroacetic acid or mixtures thereof. The carboxyalkylating agent should be added in a ratio of about 15 to about 35 wt % relative to the starch.

The mixture containing the carboxyalkylating agent is heated. In one embodiment, the mixture is heated at about 50 to about 70° C., preferably at about 65° C. The reaction should continue until gelatinization occurs. In one embodiment the mixture is heated for about 2 to about 5 hours, preferably 3 hours.

The mixture can be heated prior to addition of the carboxyalkylating agent to gelatinize the starch, but it must also be heated in the presence of the carboxyalkylating agent.

After the carboxyalkylation reaction is completed, the mixture is cooled to room temperature and the solution neutralised to about pH 6 to 7. Preferably, the final pH is about 6.6 to about 6.8. The final pH is crucial to the invention. If the pH is not lowered before recovery of the product, the product will not be water soluble nor form a gel on hydration with cold water. The cross-linked carboxyalkyl starch is then recovered from the reaction mixture, for example, by filtration. In one embodiment, the product is washed with ethanol or another polar organic solvent. The product is highly hydroscopic and water soluble, so should not be washed in water.

The product can also be dried using any suitable means known in the art, for example, air drying, oven drying, spray drying, freeze drying and the like.

### 5.4 the Gel of the Invention

The product of the process of the invention is unusual for a starch product in that it both dissolves in cold water (i.e. is cold water soluble) and also forms a gel on hydration with cold water.

To make the gel, the cross-linked carboxyalkyl starch is hydrated to form a solution which is then mixed until a gel forms. A 1% solution will generally take about 60 minutes to form a gel. Higher concentrations, (for example 2%) will need longer, for example 2-3 hours. The gel is clear and is also soluble in fresh-water.

Curiously, the gel of the invention is not suitable for use in saline solution environments, unlike some similar modified starch products. Without being bound by theory, it is thought that this may result from the absence of salts in the process by which the cross-linked carboxyalkyl starch is produced.

The gel of the invention has application as an ingredient of drilling mud. The gel of the invention can also be used in other applications, for example, as a component of a cosmetic lotion or other skin care product. Because of its high viscosity, the gel of the invention can be used in place of commercial gums such as guar gum, xanthan gum, locust bean gum carboxymethyl cellulose and the like.

The gel of the invention can also be prepared using a mixture of cross-linked carboxyalkylated starch, and other common gums such as guar gum, xanthan gum, locust bean gum, carboxymethyl cellulose and the like. The cross-linked carboxyalkylated starch of the invention is compatible with these agents and can be used to modify their properties, depending on the relative amounts of polymer used and the total polymer concentration. Example 7 describes gels made from combining the cross-linked carboxyalkylated starch of the invention with guar gum and carboxymethylcellulose.

### 6. EXAMPLES

All natural starches were obtained from National starch food innovation, New Zealand. All chemicals were supplied by Sigma-Aldrich (Bornem, Belgium). All reagents used were of at least analytical grade.

The degree of cross-linking (DCL) of the starch was determined by the method of Chatakanonda (2000). The peak viscosity was determined using a Rapid Visco Analyser (RVA). The degree of cross-linking was calculated as follows:

DCL=([A−B]/A)×100

where A is the peak viscosity of the native starch and B is the peak viscosity of the cross-linked starch.

### Example 1

### Cross-Linked Starch

1000 g of potato starch was suspended in 1500 g water. The pH of the suspension was adjusted to between 11 and 11.8 using 3% NaOH solution. Cross-linking reagents STMP or EPI were added to the reaction mixture in different concentrations (from 0.25 to 2.0% relative to the starch). The reaction mixture was stirred at room temperature for 5 hours. The reaction was terminated by lowering the pH to 5.7 with 3% HCl solution. The cross-linked starch was dried overnight at room temperature and the degree of cross-linking determined.

### Example 2

### Cross-Linked Carboxymethyl Starch

Starch was cross-linked using the procedure set out in Example 1 where 1% STMP was used. The reaction pH was 11.6 and the reaction was terminated by lowering the pH to 5.7 with 3% HCl. 30 g of the wet cross-linked starch was suspended in 80-90% ethanol. Such that the ratio of ethanol to water was between about 85:15 and 75:25 w/w. 8.0 g NaOH was added to the suspension. The reaction mixture was heated to 65° C. until gelatinization took place. Ethanol (50-100 ml) was then added. Chloroacetic acid (10.0 g) was added over 10 min and the reaction mixture maintained at 50° C. After 2 to 3.5 hrs the reaction mixture was cooled to room temperature and neutralised to pH 7 with acetic acid. The carboxymethyl cross-linked starch product was recovered by filtration using a glass sinter, washed with ethanol and then dried under vacuum.

### Example 3

### Rheological Properties of Cross-Linked Carboxymethyl Starch Compared to Commercial Gums

Cross-linked carboxymethyl starch was prepared in accordance with Example 2. The starch was cross-linked using 1% STMP and reacted with chloroacetic acid for 3.5 hours.

The modified starch was then hydrated in water and stirred until a gel (or gum) formed.

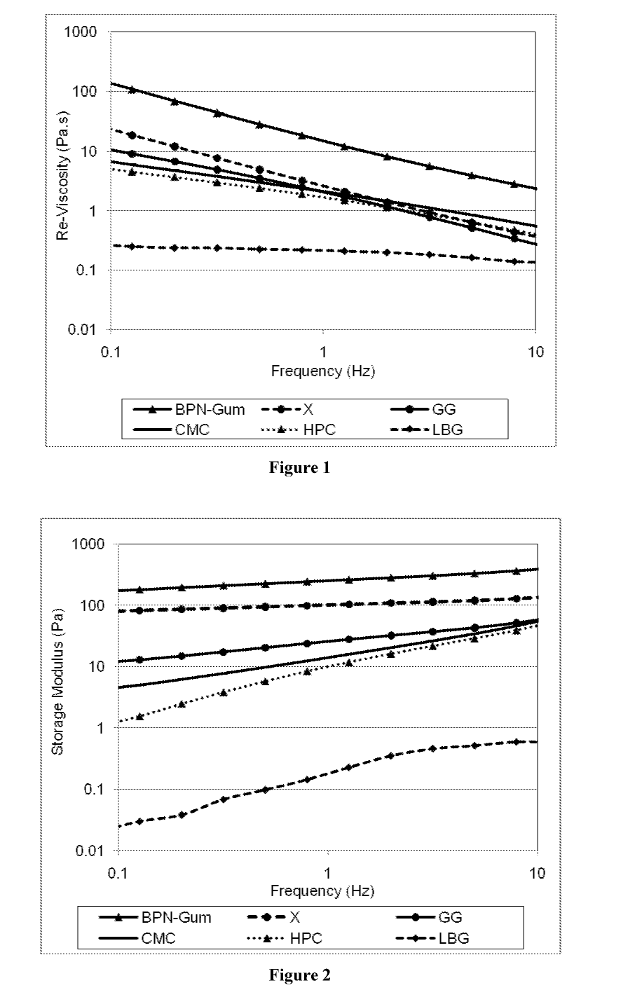

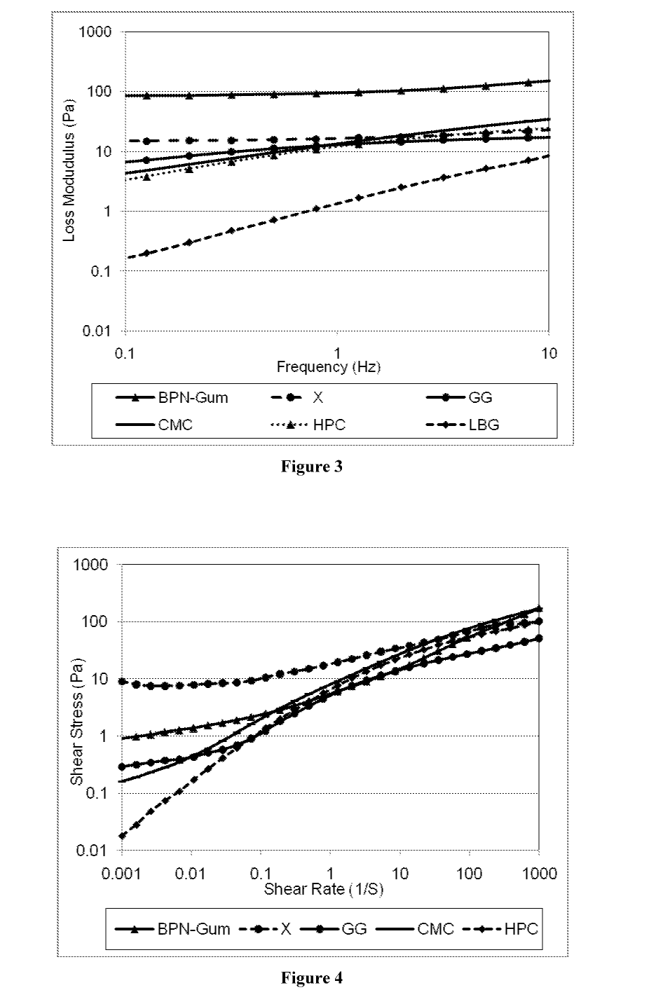

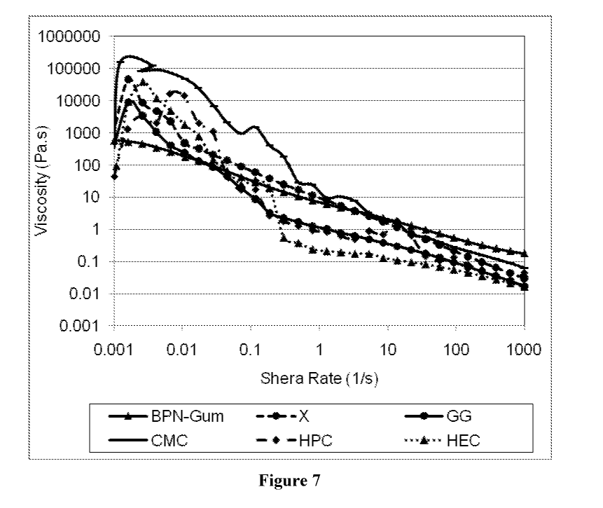

The rheological properties of this material (designated BPN-Gum) were compared with some of the most commonly used commercial gums available in the market, as shown in FIGS. 1 to 7. The other gums used were Xanthan gum (X), Guar gum (GG), Locust bean gum (LBG), Carboxymethyl cellulose (CMC), Hydroxy propyl Cellulose (HPC)

As shown in FIGS. 1 to 3, the relative viscosity, storage modulus and loss modulus of BNP-Gum was higher than that of any of the other gums.

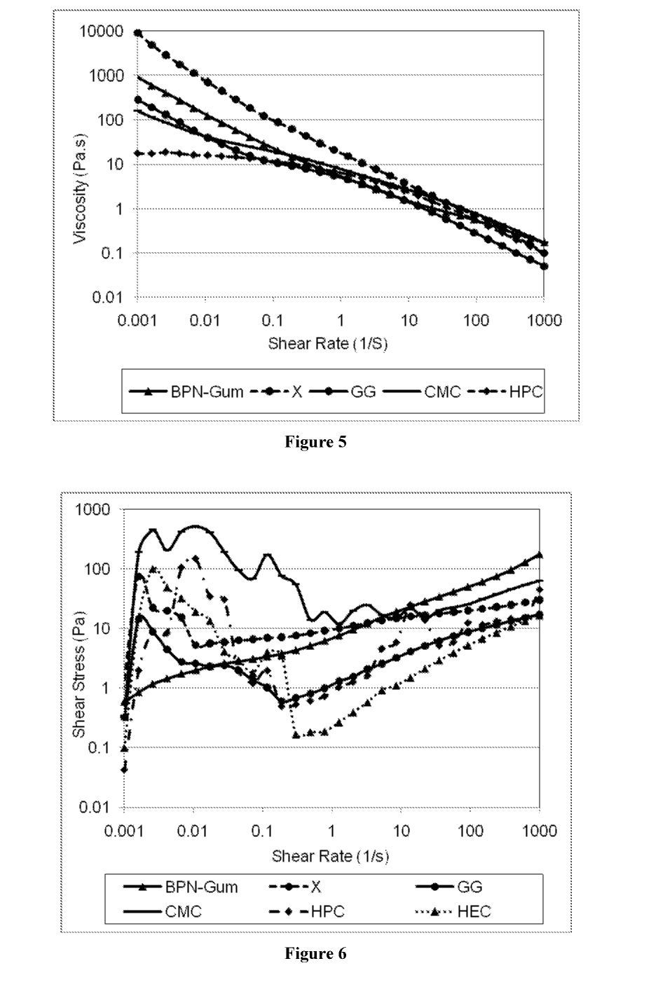

FIGS. 4 and 5 illustrate the flow behaviour of BPN Gum at 20° C. compared to different commercial gums. The viscosity of BPN-Gum during changes of shear rate demonstrated shear thinning behaviour, which was compatible with most of the commercial gums used in this study. Only Xanthan gums showed slightly higher viscosity at the start of the shear during the flow study.

The flow behaviour of the commercial gums at 75° C. showed a very different profile, with big fluctuations at this high temperature. On the contrary, the BPN-gum showed steady state during the shearing that took place during the flow study at 75° C.

### Example 4

### Evaluation of Gum Samples for Effectiveness in Filtration Control of Drilling Fluids in Oil Well Drilling

Tests were carried out at the London South Bank University using a standard API filter press procedure at low pressure (100 psi and room temperature) and under high pressure and high temperature (HPHT) conditions (500 psi and 100° C.). The fluid loss and filter cake thickness were determined.

The base mud was prepared using 6.0% bentonite slurry. Filtration control agents were added to the slurry including commercial xanthan gum (Cl) and a gel comprising the cross-linked carboxymethyl starch of the invention (BNP-gum) prepared as described in Example 3. The experiments were performed using deionised water. The results are shown in Table 1 below.

TABLE 1

| | API Filter press | | HPHT conditions | |
|---|---|---|---|---|
| | Filtrate | FC thickness | | FC thickness |
| The mud | (ml) | (mm) | Filtrate (ml) | (mm) |
| 6.0% wt Bentonite | 23.0 | 4.1 | 43.0 | 6.5 |
| Slurry (Base Mud) | | | | |
| +C1 (0.1% wt) | 12.0 | 1.6 | 27.0 | 4.1 |
| +BNP-gum | 14.0 | 1.6 | 28.0 | 5.3 |
| (0.1% wt) | | | | |
| +Polymer (0.5% wt) | 10.5 | 1.0 | 25.0 | 3.4 |
| +Polymer (0.5% wt) | 10.0 | 0.9 | 23.0 | 3.4 |
| and C1 (0.1% wt) | | | | |
| +Polymer (0.5% wt) | 9.5 | 1.2 | 24.0 | 3.0 |
| and N1 (0.1% wt) | | | | |

One of the roles of the bentonite, gum and starch components added to drilling mud is to reduce fluid loss during the drilling process. The addition of these components reduces fluid loss from the drilling mud and also influences the properties of the filter cake.

During drilling, the drilling fluid forms a deposit on the side of the drilling well. This deposit is called a “filter cake”. The filter cake helps prevent the drilling fluid from entering the walls of the well, which would destabilise them. Generally, a thin, tough filter cake is preferred. As the results of Table 1 show, the fluid loss from the base mud is greatly reduced when both xanthan gum and BNP-gum are added to a bentonite-containing base mud. This occurs under both standard (API filter press) and HTHP conditions.

A similar improvement is also seen when a commercial synthetic filtration control polymer is also added to the base mud.

The thickness of the filter cake is also greatly reduced when either xanthan gum or BNP-gum is added to the base mud, under both types of conditions.

These results demonstrate that the cross-linked carboxymethyl starch of the invention (BNP-gum) forms a gel that is surprisingly suitable for use as an additive in drilling muds.

### Example 5

100 g starch was dispersed in 350 ml water. NaOH solution (3%) was added to bring the pH to 11.6. The suspension was stirred for 20 min then STMP (1.0-1.2 wt % based on the starch) added. The reaction mixture was stirred at room temperature for 5 hours.

The product was filtered to produce a wet cross-linked starch (135 g, water content approximately 35%). The filtered product was placed in a beaker and 95% ethanol added to give an ethanol to water ratio of about 80:20 v/v. NaOH (26 g) was added and the mixture heated to about 65° C. with stirring. Chloroacetic acid (32 g) was added and the mixture stirred for about 3 hours. The reaction mixture was then cooled to room temperature and neutralised with HCl to pH about 6.5. The product was filtered and washed with 80% ethanol several times, then 95% ethanol.

The cross-linked carboxymethyl starch product was then dried under vacuum. The product, when hydrated with cold water (1%), formed a clear gel.

### Example 6

The process of example 5 was repeated using EP 1 as the cross-linkeding reagent. The product, when hydrated with cold water, (1%) formed a clear gel.

### Example 7

Mixed gels were prepared using the cross-linked carboxymethylated starch of the invention and (a) guar gum and (b) carboxymethyl cellulose. The product of the invention was mixed with either (a) or (b) in ratios of 80:20, 60:40 and 25:75 then hydrated. The mixed material was used to prepare gels at various concentrations (0.5%, 1%, 1.25%, 1.50%, 1.75% and 2%).

### 7. INDUSTRIAL APPLICABILITY

The cross-linked carboxyalkyl starch product of the invention forms a gel in cold water that can greatly increase the viscosity of a liquid when a small amount is added to said liquid. This ability is also seen in natural gums such as xanthan, locus bean and guar gums. These gums are used in a wide range of industries in many different applications. The cross-linked carboxyalkyl starch product of the invention offers a superior cost-effective alternative to these products.

1. A cross-linked carboxyalkyl starch that forms a gel on hydration with cold water.

2. A cross-linked carboxyalkyl starch that forms a gel on hydration with cold water when mixed in water at a concentration of about 1 wt %.

3. The cross-linked carboxyalkyl starch of claim 1 which is prepared by cross-linking a native starch and has a relative viscosity that is more than 100 times greater than the relative viscosity of the native starch.

4. The cross-linked carboxyalkyl starch of claim 1 which is prepared by cross-linking a native starch and has a storage modulus G′ that is more than 100 times greater than the G′ of the native starch.

5. The cross-linked carboxyalkyl starch of claim 1 which is prepared by cross-linking a native starch and has a loss modulus G″ that is more than 50 times greater than the G″ of the native starch.

6. The cross-linked carboxyalkyl starch of claim 1 which is cross-linked carboxymethyl starch.

7. A process for making a cross-linked carboxyalkyl starch that forms a gel on hydration with cold water, the process comprising the steps of:
(a) adding a cross-linking reagent to a suspension of starch in water at about pH 11.0 to 11.8;
(b) stirring the suspension at about room temperature until cross-linking has occurred, wherein the suspension is substantially free of anti-gelatinization salts;
(c) removing the solvent to produce a wet cross-linked starch product comprising about 25 to about 40 wt % water;
(d) mixing the wet cross-linked starch product with a C₁-C₃ alcohol at pH above about 11 such that the ratio of C₁-C₃ alcohol to water is about 90:10 to about 70:30 v/v;
(e) heating the mixture to about 50° C. to about 70° C. with one or more carboxyalkylating agents in a ratio of about 15 to about 35% w/w relative to the starch, until gelatinization occurs;
(f) cooling to room temperature and neutralising the solution to about pH 6 to 7;
(g) recovering the cross-linked carboxyalkyl starch product from the reaction mixture; and
optionally, drying the product to provide a powder

8. The process of claim 7 wherein the cross-linking reagent is selected from the group consisting of sodium trimetaphosphate (STMP), sodium tripolyphosphate (STPP), epichlorohydrin (EPI) and mixtures thereof.

9. The process of claim 7 wherein the pH of the suspension of starch of step (a) is about 11.6.

10. The process of claim 7 wherein the wet cross-linked starch product is mixed with ethanol at above pH 12.

11. The process of claim 7 wherein the carboxalkylating agent is chloroacetic acid or a mixture of chloroacetic acid and trichloroacetic acid.

12. A cross-linked carboxyalkyl starch produced according to the process of claim 7.

13. A gel comprising a cross-linked carboxyalkyl starch of claim 12.

14. The gel of claim 13 which comprises about 0.5 to 1.5 wt % of the cross-linked carboxyalkyl starch.

15. The gel of claim 13 which is clear.

16. A drilling method, comprising drilling with a drilling fluid which comprises the gel of claim 13.

17. A method of manufacturing a cosmetic lotion or skin care product, comprising preparing a cosmetic lotion or skin care product that comprises the gel of claim 13.

18. (canceled)
